(19) United States
(12) Patent Application Publication
(10) Pub. No.: US 2006/0135645 A1
(43) Pub. Date: Jun. 22, 2006

(54) Pest protection methods and compositions

(76) Inventors: David Glassel, Spring, TX (US); Gary Ernest Mellema, Grapevine, TX (US)

Correspondence Address: Martin L. McGregor, 26415 Oak Ridge Drive, Spring, TX, 77380, US

(21) Appl. No.: 11/303,263

(22) Filed: Dec. 15, 2005

Related U.S. Application Data
(63) Continuation-in-part of application No. 11/016,584, filed on Dec. 17, 2004.
(63) Continuation-in-part of application No. 11/016,627, filed on Dec. 17, 2004.

(51) Int. Cl. C09D 5/16; A61K 36/736; A61K 36/752; A61K 36/53; A61K 36/534; A61K 36/73; A61K 36/13

(52) U.S. Cl. 523/122; 424/725; 424/745; 424/747; 424/770; 424/778; 424/736; 424/735; 424/765

(57) ABSTRACT

A method and composition for the method for preventing insect infestation that comprises applying to the area to be protected an effective amount of solid material coated with by treatment with a composition comprising a silicone polymer, and an essential oil. A protective effect by reducing the population of harmful biological pests in the locality to be protected is provided. Protection by penetrating the material to be protected with the silicone polymer and optionally an essential oil is also disclosed.

### RELATED APPLICATIONS

This application is a continuation in part of U.S. patent application Ser. Nos. 11/016,584 and 11/016,627 both filed Dec. 17, 2004.

### TECHNICAL FIELD

This invention provides methods and compositions of matter for protecting against biological pests. The methods and compositions of the invention are especially useful in protecting growing plants and buildings against insects by applying an essential oil coated on a solid carrier. Alternatively the composition of the invention with an added hydrocarbon carrier may be used to treat materials to be protected against pests by penetration. In another alternative moisture is excluded by application of the silicone polymer with a carrier to facilitate penetration.

### BACKGROUND OF THE INVENTION

Repealing insects by treatment with various agents have been practiced for many years. The products normally used for repealing insects generally comprise one or more active substances that are toxic to at least some organisms.

This invention relates to insect, plant, or microbial control compositions and methods of using same to control various crawling and flying insect pests, and, in its preferred embodiments, relates more particularly to “non-poison” insecticides adapted to increase insect protection by repellent qualities as well as increasing mortality. Commercially available insecticides, including those available for home use, commonly comprise active ingredients or “poisons” which are not only toxic to the target insect pests, but, if used in relatively confined environments and delivered as aerosol sprays, can be present in sufficient concentration to also be toxic to humans and household pets. Various undesirable side effects may include immediate or delayed neurotoxic reactions, and/or suffocation. Even the noxious odor of such materials can cause headaches or upset stomachs in some individuals. These adverse side effects are exacerbated when such compositions come in contact with persons of increased sensitivity, or persons of small body mass such as children or babies. For some time, efforts have been made to develop insecticidal compositions, particularly those intended for residential use in aerosol form, which are effective in killing the targeted insect pests completely and quickly, but non-toxic to humans and pets. It has now been found to be even more desirable simply to deter insect infestation because high kill rates accelerate the development of immune insect populations. The Environmental Protection Agency (EPA) regulates the use of potentially toxic ingredients in pesticidal compositions under the Federal Insecticide, Fungicide and Rodenticide Act. Certain materials considered either active or inert materials by the EPA have been deregulated or otherwise identified as acceptable “safe” substances offering minimum risk in normal use. Other materials are currently undergoing investigation and may be deregulated in due course. Deregulated substances are generally considered non-poisonous by the consumer. Thus, the term “non-poisonous” as used herein is intended to convey a composition that, while highly effective in killing or repelling targeted insect pests, is safe to use around humans, particularly small children, and pets. Non-poisonous insecticidal compositions available heretofore incorporating deregulated materials as the active ingredient were believed to have had limited efficacy due to low kill rates, the benefits of repellents were vastly under rated. Attempts to use deregulated essential oils as the active ingredient in such insecticides, while having limited success, have generally been found to be either cost prohibitive, inadequately lethal to control a range of targeted insect pest species, or too slow-acting to enable the user to confirm that the insect has been killed and to dispose of the dead insect so as to avoid polluting the environment. Among the insects which are found to be particularly undesirable are cockroaches, both the American and German species. These pests shed their “skin” which, over time, disintegrates forming what is known as “cuticle” in the air, a particular problem for people suffering from asthma. Thus, not only is it important to kill cockroaches with an effective insecticide, the kill time must be sufficiently short for the carcass to be properly disposed of before the insect can crawl into a remote area to die. While cockroaches are a prime target for a household spray, for general application, such materials must also be effective against other crawling pests, such as ants, water bugs, silverfish, crickets, spiders and centipedes. Additionally, aerosol compositions of such insecticides of proper concentration must also be effective against various flying insects, including flies, mosquitoes, gnats, moths, wasps, hornets, yellow jackets, bees and other flying insects, both inside and outside of the house. Essential oils currently deregulated by the EPA include cedar oil, cinnamon oil, citronella oil, clove oil, corn oil, garlic oil, lemongrass oil, linseed oil, peppermint oil, rosemary oil, soybean oil and thyme oil. Among the essential oils proposed for exemption from registration are a number of the citrus oils. Citrus oils would include orange oil, lemon oil, lime oil, grapefruit oil and tangerine oil. Some of these other essential oils have been considered for their insecticidal or insect repellent properties, but they have not been shown to be particularly effective as active ingredients. In contrast, cedar oil, citronella oil, clove oil, peppermint oil and others have long been recognized as highly effective. Therefore, it would be both environmentally and commercially important to be able to enhance the properties of these relatively safe essential oils by a formulation and application method that provides a protective effect in a cost effective manner. It is also desirable to select the particular active ingredient to protect against the indigenous insect populations in a particular location. For many applications, it has been found to be especially desirable to combine an essential oil with a silicone polymer and a solid carrier. It is especially convenient to use locally available natural materials such as solid agricultural wastes, for example corn cob prills, as a carrier.

Preservation of construction materials by treatment with various agents has been practiced for many years. Among the earliest treatments was the application of tars or creosotes to wood such as railway ties that would be in contact with the earth. Various harmful biological agents are known that are capable of attacking wood, causing significant damage. Among these biological agents harmful to wood are insects, for example beetles (a term which includes various species of wood-eating insects whose larvae gnaw and bore wood), termites, moths, etc., and fungi, for example Poria vaillantii Fr., Coniophora cerebella Duey, Merulius lacrymans Wulf, Lentinus lepideus Fr., Lenzites sepiaria Wulf, Chaetomium globosum Kunz, etc. Many of these species are known to favor wet or water soaked wood. The products normally used for treating and conserving wood generally comprise one or more active substances that are effective against the harmful biological agents to be eliminated, for example one or more insecticides and/or fungicides, and a vehicle for introducing the active substances into the tunnels and cavities generated in the wood by the action of the harmful biological agents. The number of vehicles that are effective as active substance carriers inside the wood is currently very limited. Among those materials normally used in products for treating and conserving wood are water, inorganic arsenic derivatives, pentachlorophenol and creosote. Water is the most commonly used carrier for wood preservatives. However, it scarcely penetrates the wood after application and allows diffusion of materials from the treated wood into the environment. Furthermore the active substances applied to the wood using water as the vehicle often do not satisfactorily penetrate it, remaining on the surface, thereby significantly reducing the effectiveness of the active substances. Ramos, U.S. Pat. No. 6,673,836 states that “other vehicles have a very slight wood penetration index, generally about 2 to 6 mm after 24 hours. In soft and porous woods, penetration indices of the vehicle of up to 6 mm are obtained, whereas in hard and non-porous woods, the vehicles penetrate between 2 and 3 mm during a 24 hour time period.” Ramos disclosed many combinations that were considered of little use and claimed a mixture of toluene (40-70%), xylene (6-40%), benzophenone (3-18%), butyl glycol (2-9%), cetyl acetate (1-7%) and methanol (0.3-4%), as a carrier for insecticides into wood. Ramos clearly teaches away from the non-aromatic materials used in the present invention. In contrast to Ramos results, the present invention completely penetrates the wood. Tests have been conducted up to the size of railway crossties with complete penetration from one hour of immersion.

A method of and a composition for treating wood with an insecticide is disclosed in Qader, U.S. Pat. No. 6,638,574. The composition comprises an insecticide such as a pyrethroid dissolved in a supercritical fluid such as carbon dioxide. The composition may also include a cosolvent such as methanol. The method includes impregnating wood with the composition and reducing the temperature and pressure below critical levels to precipitate the insecticide within the wood. In contrast to Qader's approach that requires use of pressure vessels to maintain supercritical solvent conditions, the methods of the present invention can be practiced at ambient temperature and pressure. Again the resistance to penetration noted by Qader is surprisingly overcome by the compositions of the present invention. A secret formula wood preservative mixture claiming insect protection was widely marketed in the United States under the trade names Seasonal and Vaccinol from the 1920's to the late 1950s. Whatever this unknown material contained it is certain that it did not contain the silicone materials of the present invention which were not discovered until decades later.

### SUMMARY OF THE INVENTION

The invention provides a composition of matter useful for preventing biological pest infestations that comprises a solid and a biologically effective amount of an oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil, or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil mixed with a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable and a crosslinking catalyst wherein the base copolymer is 75 to 90% by weight and the crosslinker is 9 to 24% by weight and the catalyst is 1 to 5% by weight of the component. The core may be any solid material, for example it may be a particulate clay, expanded clay, diatomaceous earth, glass beads, silica gel beads, zeolites, soil amendments, such as gypsum or calcium sulfate particles, lime, and the like, nontoxic spent catalyst waste; fertilizer, time release fertilizer pellets, grain casings such as, rice hulls, wheat chaff and the like, nut shells such as peanut hulls, pecan shells, almond shells, walnut shells, pistachio shells, hazelnut shells, and the like, wood particles such as pelletized wood dust, sawdust, wood chips, wood shavings bark mulch, vegetative matter such as processed soy bean meal, dried vegetable wastes, pelletized vegetative matter, coir, corn cob prills, mulch, and the like. Preferably, the foregoing mixture is applied as a coating to an absorbent plant material as a particulate carrier, the currently most preferred core material is corncob prill. The particle size may be any size that is convenient for application, however the more finely divided the material, the better it is expected to perform so long as it is not so finely divided and as to not settle when applied. Preferably, the size range is the same as that used for other solids applied to growing plants such as soil amendments, mulches or fertilizers. Preferably, the coating composition is from 1 to 99% preferably 70 to 90% essential oil, more preferably about 80% essential oil and the residue is substantially the silicone polymer. The composition preferably includes an oil selected from the group consisting of cedar oil, cinnamon oil, citronella oil, clove oil, eucalyptus oil, juniper oil, tall oil, and pine oil especially preferred is cedar oil, also known as cedar wood oil.

The invention may also be considered as a method of treatment useful for protecting an area against insect infestation that comprises applying an effective amount of a particulate solid material with a coating comprising a mixture of the following components: (1) at least a biologically effective amount of an oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and (2) at least 10% of a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and a crosslinking catalyst wherein the base copolymer is 75 to 90% by weight and the crosslinker is 9 to 24% by weight and the catalyst is 1 to 5% by weight of the component. Preferred natural oils include cedar oil, cinnamon oil, citronella oil, clove oil, eucalyptus oil, juniper oil, tall oil, and pine oil, cedar oil (also known as cedar wood oil) is especially preferred. Preferred carriers are materials that are normally applied to growing plants such as mulches, fertilizers, soil amendments, and the like, or materials that may be readily mixed with such materials such as a corncob prill.

The invention may also be considered as a composition useful in practicing the method of the invention that comprises a plant material coated with a mixture comprising the following components: (1) a biologically effective amount in the range or 1 to 99% by weight of a natural product oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and (2) the balance comprising a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and a crosslinking catalyst. Preferably, the mixture may also comprise one or more agricultural adjuvants such as surfactants and the like. Preferred compositions are those wherein the base copolymer is 75 to 90% by weight and the crosslinker is 9 to 24% by weight and the catalyst is 1 to 5% by weight of the component and the overall mixture is from 65 to 95% by weight aliphatic solvent, a biologically effective amount of an oil, preferably from 1 to 80% by weight oil and the silicone based polymer is at least 5% by weight. The composition also comprises a catalyst that promotes film formation in the silicone-based component. While any catalyst may be used preferred catalysts are metal soaps, especially preferred are crosslinking catalysts selected from the group consisting of metal salts of alkylcarboxylic acids having from 2 to 18 carbons, and more especially preferred metal soaps are tetraalkyl titanates or zirconates.

The invention may also be considered to be a method of preparing a biologically active plant protection composition comprising the steps of mixing a particulate solid material in a chamber and spraying the solid during the mixing of the solid with a mist of a composition that comprises (1) a quantity of a selected oil sufficient to provide a biologically effective amount in the finished product of at least one oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; or a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and the balance further comprising at least one silicone based polymer that forms a film in the presence of a catalyst and water comprising at least one component selected from the group consisting of (A) a copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; and (B) (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group and (C) a crosslinking catalyst and curing the mixture in the presence of water to provide a free flowing particulate solid material.

The invention provides a composition of matter useful for preventing damage to construction materials containing cellulose fiber by biological agents by adding a hydrocarbon carrier and replacing the core material with a material to be protected. The composition comprises from 65 to 99% of a high-purity, low-odor aliphatic solvent composed primarily of C₇-C₂₀ straight chained paraffinic, cycloparaffinic and isoparaffinic hydrocarbons, that contains less than 0.5% aromatics, a biologically effective amount of an oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil, and at least 5% of a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable and a crosslinking catalyst wherein the base copolymer is 75 to 90% by weight and the crosslinker is 9 to 24% by weight and the catalyst is 1 to 5% by weight of the component. Preferably, the aliphatic solvent is composed primarily of C₉-C₁₄ cycloparaffinic and isoparaffinic hydrocarbons more preferably the aliphatic solvent is composed primarily of C₁₀-C₁₃ cycloparaffinic and isoparaffinic hydrocarbons. The best aliphatic solvent known to the inventors is composed primarily of a Conosol solvent designated as Conosol 145 by its manufacturer, Penreco, Inc. The composition preferably includes an essential oil and preferred oils are selected from the group consisting of cedar oil, cinnamon oil, citronella oil, clove oil, eucalyptus oil, juniper oil, tall oil, and pine oil especially preferred is cedar oil, also known as cedar wood oil.

A preferred method of treatment useful for improving the properties of wood for use as a construction material comprises contacting wood with a mixture of the following components: (1) at least 70% by weight of an aliphatic solvent composed primarily of C₇-C₁₆ straight chain aliphatic, cycloparaffinic and isoparaffinic hydrocarbons, that contains less than 0.5% aromatics; (2) 1 to 5% by weight of a natural product oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and (3) at least 10% of a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable and a crosslinking catalyst wherein the base copolymer is 75 to 90% by weight and the crosslinker is 9 to 24% by weight and the catalyst is 1 to 5% by weight of the component. Preferred natural oils include cedar oil, cinnamon oil, citronella oil, clove oil, eucalyptus oil, juniper oil, tall oil, and pine oil, cedar oil (also known as cedar wood oil) is especially preferred.

The invention may also be considered as the composition useful in the In practicing the method of the invention that comprises a mixture of the following components: (1) at least enough aliphatic hydrocarbon solvent composed primarily of C₇-C₁₆ straight chain aliphatic, cycloparaffinic and isoparaffinic hydrocarbons, that contains less than 0.5% aromatics; (2) a biologically effective amount not to exceed about 5% of a natural product oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and (3) a silicone based polymer that comprises a mixture of (A) a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; (B) a crosslinker having a general (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable and a crosslinking catalyst. Preferred compositions are those wherein the base copolymer is 75 to 90% by weight and the crosslinker is 9 to 24% by weight and the catalyst is 1 to 5% by weight of the component and the overall mixture is from 65 to 95% by weight aliphatic solvent, a biologically effective amount of a natural product oil, preferably from 1 to 5% by weight oil and the silicone based polymer is at least 5% by weight. The composition also comprises a crosslinking catalyst that promotes film formation in the silicone-based component. While any catalyst maybe used preferred catalysts are metal soaps, especially preferred are catalysts selected from the group consisting of metal salts of alkylcarboxylic acids having from 2 to 18 carbons, and more especially preferred metal soaps are tetraalkyl titanates or zirconates.

Protection of wood and other cellulose based materials against fungi and insects can also be achieved by exclusion of absorbed water from wood fiber or cellulose materials by contacting the surface to be protected with a reactive silicone polymer having the following characteristics: a base copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group. Preferred polymers are those having R¹ groups that are methyl and R² groups that are not methyl, where 70 to 99% of the groups are R¹ and 1% to 10% of R² groups have a hydroxyl, alkoxy, or acyl group. The natural viscosity of the silicone polymer limits its entry into a cellulose fiber or wood vascular system. While surface treatment is possible with the undiluted polymer, penetration of a material requires thinning of the polymer. Therefore it is helpful to dilute the silicone polymer with a diluent to lower its viscosity and enhance vascular mobility of the polymer. A suitable diluent will not be damaging to the environment or to humans or pets exposed to the treated wood. White mineral oils, particularly an aliphatic solvent composed primarily of C₇-C₁₆ straight chain aliphatic, cycloparaffinic and isoparaffinic hydrocarbons, that contains less than 0.5% aromatics is an effective diluent for silicone polymers. Especially preferred are an aliphatic solvent composed primarily of C₉ to C₁₄, more preferably C₁₀-C₁₃ straight chain paraffinic, cycloparaffinic and isoparaffinic hydrocarbons, that contains less than 0.5% aromatics, such as Conosol 145 marketed by Penreco, Inc. of Houston Tex. A suitable composition will have at least 5% silicone polymer and the balance will be a diluent.

### DETAILED DESCRIPTION OF THE INVENTION

General Description of the Invention

Definitions

A “biologically effective amount of an oil” means enough material is present to produce a statistically significant increase in protection against at least one biological pest over a control treated with substantially the same materials but for the biologically active oil. Unless otherwise noted all terms used herein have the meaning normally ascribed in the arts that use the terms in the normal course of practice in that art. A biological effect may be a lethal effect or a non-lethal effect such as repelling an insect or inhibiting feeding or inhibiting the growth of a pest plant or micro-organism or interrupting the life-cycle of a pest organism or deterring spore formation or attracting to the area or sustaining the growth of a beneficial insect or micro organism in the area to be protected.

### EXAMPLE 1

Preparation of Compositions of the Invention

The compositions of the invention comprises a particulate core, preferably a material normally applied to growing plants, more preferably an absorbent plant material such as a mulch or a corn cob prill, the core being coated with an essential oil in a silicone polymer that is a mixture of alkylsiloxanes having a general base formula of: (MₐD_{b}T_{c}Q_{d})ₓ where M is R₃SiO_{1/2}—; D is R₂SiO—; T is RSiO_{3/2}—; and Q is Si(O_{1/2})₄— and R is a generalized organic radical selected from: linear or branched hydrocarbon radicals of 1-8 carbons containing 0-1 degree of unsaturation, or phenyl, or trifluoropropyl radicals, and may optionally be substituted with a hydroxyl, alkoxy or acyloxy group of 1 to 8 carbons. A preferred embodiment is ^{HO}MDₓM^{OH} namely a silanol endblocked polydimethylsiloxane. The preferred viscosity is 50-3500 cSt with 750-1500 cSt being preferred. The composition is subject to the following general parameters: The ratio of a/(c+d) is between 0 and 4 with the preferred range being 0-0.5.

The ratio of b to the rest is not subject to limitation provided the final base viscosity is between 50-3500 cSt with 750-1500 being preferred. R at each position may be the same or different and will be predominately methyl. All R groups being methyl is a preferred choice. In addition, at least one R group of each molecule must include a hydrolysable group such as hydroxy, alkoxy or acyloxy with hydroxy being preferred. The silicone polymer may include a further component capable of crosslinking of the general formula (MₐD_{b}T_{c}Q_{d})ₓ where M, D, T and Q are as defined above and meeting the following parameters: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group. The silicone polymer may also comprise mixtures of the polymer and the crosslinker, and may further comprise a catalyst. Preferred silicone polymers form films in the presence of moisture. Any catalyst that promotes crosslinking may be used. Preferred catalysts are metal soaps, especially preferred are tetraalkyl titanate and tetraalkyl zirconates.

The preferred silicone polymers comprise from 75 to 90% of base polymer more preferably 80 to 85%, most preferable about 82.6% and from 10 to 25% crosslinker more preferably 10 to 17%, most preferably 15% and from 1 to 5% of a catalyst preferably 2 to 3% and most preferably 2.4%. The presently preferred silicone polymer is available from GT Products, Grapevine Tex. as GT 5814.

The essential oil is combined with the silicone polymer. The essential oil is selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil. Preferred oils are cedar oil, cinnamon oil, citronella oil, clove oil, eucalyptus oil, juniper oil, tall oil, and pine oil. Cedar oil (also to known as cedar wood oil) is especially preferred. Preferred cedar oil is available from CedarCide, Inc. of Spring, Tex.

The solid material may be any material convenient to the area where the product will be formulated and that is easily processed into a particulate material that is preferably finely divided and easily handled. Such material may include plant stems having a pithy core such as native North American marsh plants of the species hibiscus, such as H. moscheutos, H. moscheutos spp. palustris, and H. laevis (formerly H. militaris), and H. coccineous. An especially desirable plant material is corncobs ground to the composition known in the trade as “prill”. The core may also be particulate clay, expanded clay, diatomaceous earth, glass beads, silica gel beads, zeolites, soil amendments, such as gypsum or calcium sulfate particles, lime, and the like, nontoxic spent catalyst waste; fertilizer, time release fertilizer pellets, grain casings such as, rice hulls, wheat chaff and the like, nut shells such as peanut hulls, pecan shells, almond shells, walnut shells, pistachio shells, hazelnut shells, and the like, wood particles such as pelletized wood dust, sawdust, wood chips, wood shavings bark mulch, vegetative matter such as processed soy bean meal, dried vegetable wastes, pelletized vegetative matter, coir, corn cob prills, mulch, and the like.

The composition is prepared as follows: In a power stirred vessel is placed a volume of oil and the silicone-based polymer is slowly added with stirring. In this manner compositions of 80% Cedar 20% GT 5814; 75% Cedar oil/25% GT 5814; 65% Cedar oil/35% GT 5814 and 60% Cedar oil/and 40% GT 5814 are prepared. Samples of commercially available corncob prills are treated by aerosol spraying tumbled prills with a commercial paint spray gun powered by compressed air at 200 psi. Compositions containing a biologically effective amount of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil are prepared in a similar manner as that described above for cedar oil.

The invention is highly effective in treating gardens and forming a protective zone around dwellings and other buildings having landscaping substantially surrounding the structure. The exact selection of treatment compositions and materials will vary depending on the specific protection needed and the availability of treatment materials near the source of the place to be protected.

### EXAMPLE 2

Protection Against Insects

Prills comprising corncob particles were sprayed with a composition prepared as set out in example 1 and comprising 80% cedar oil 20% GT 5814 allowed to dry. An untreated control was also provided. The prills were placed in a test chamber and various insects collected in Spring Tex. were placed in the chamber. In each case after 24 hours, the insects were dead. Ten species of insects native to south Texas including German cockroach, cat fleas, mosquitoes, gypsy moths Japanese beetles, horned beetles, were placed in the glass aquarium and exposed to 10 grams of prills treated with a composition prepared as set out in example 1. As noted above after 24 hours, in each case the insects in the treated chamber were dead while those in the control chamber having untreated prill were active.

Protecting Other Materials

The protective effects of the invention arise from carrying the biologically active agent into the micro-pores of the wood with a reactive silicone polymer that improves retention of the oil by the wood fibers and lignins within the micropores. The stabilization of the wood's moisture content at less than 20% and reduction in the ability of the wood fibers to absorb water is in itself an important protective effect against biological attack. The silicone polymer and biologically active materials are carried into the wood by the natural vascular system of the wood. As the carrier penetrates into the vascular system, the silicone polymer alters the wood surfaces it contacts to render the wood less hydrophilic and to facilitate movement of the protecting oil deeper into the wood and its retention within the wood.

### EXAMPLE 3

Preparation of Compositions with Hydrocarbon Carrier

The compositions of the invention comprise a silicone polymer that is a mixture of alkylsiloxanes having a general base formula of: (MₐD_{b}T_{c}Q_{d})ₓ Where M is R₃SiO_{1/2}—; D is R₂SiO—; T is RSiO_{3/2}—; and Q is Si(O_{1/2})₄— and R is a generalized organic radical selected from: linear or branched hydrocarbon radicals of 1-8 carbons containing 0-1 degree of unsaturation, or phenyl, or trifluoropropyl radicals, and may optionally be substituted with a hydroxyl, alkoxy or acyloxy group of 1 to 8 carbons. A preferred embodiment is: ^{HO}MDₓM^{OH} namely a silanol endblocked polydimethylsiloxane. The preferred viscosity is 50-3500 cSt with 750-1500 cSt being especially preferred. The composition is subject to the following general parameters: The ratio of a/(c+d) is between 0 and 4 with the preferred range being 0-0.5. The ratio of b to the rest is not subject to limitation provided the final base viscosity is between 50-3500 cSt with 750-1500 being preferred. R at each position may be the same or different and will be predominately methyl. All R groups being methyl is a preferred choice. In addition, at least one R group of each molecule must include a hydrolysable group such as hydroxy, alkoxy or acyloxy with hydroxy being preferred. The silicone polymer may include a further component capable of crosslinking of the general formula (MₐD_{b}T_{c}Q_{d})ₓ where M, D, T and Q are as defined above and meeting the following parameters: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable group. The silicone polymer may also comprise mixtures of the polymer and the crosslinker, and may further comprise a catalyst. Preferred silicone polymers form films in the presence of moisture. Any catalyst that promotes crosslinking may be used. Preferred catalysts are metal soaps, especially preferred are tetraalkyl titanate and tetraalkyl zirconates.

The preferred silicone polymers comprise from 75 to 90% of base polymer more preferably 80 to 85%, most preferable about 82.6% and from 10 to 25% crosslinker more preferably 10 to 17%, most preferably 15% and from 1 to 5% of a catalyst preferably 2 to 3% and most preferably 2.4%. The presently preferred silicone is available from GT Products, Grapevine Tex. as GT5814.

The silicone is diluted with an aliphatic solvent composed primarily of C₇-C₁₆ straight chain aliphatic, cycloparaffinic and isoparaffinic hydrocarbons, that contains less than 0.5% aromatics. Preferably the aliphatic solvent is selected is composed primarily of C₉-C₁₄ cycloparaffinic and isoparaffinic hydrocarbons, more preferably primarily of C₁₀-C₁₃ cycloparaffinic and isoparaffinic hydrocarbons, another preferred the aliphatic solvent is composed primarily of solvents capable of meeting applicable standards for a “food grade” classification. The currently most preferred solvent is Conosol 145 marketed by Penreco, Inc. of Houston, Tex. Other suitable solvents are available from Shell Oil Company under the name Shellsol.

Optionally a natural product oil may also be combined with the silicone polymer. The oil may be selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil. Preferred oils are cedar oil, cinnamon oil, citronella oil, clove oil, eucalyptus oil, juniper oil, tall oil, and pine oil. Cedar oil (also known as cedarwood oil) is especially preferred.

The over all composition requires a silicone polymer as described above with sufficient aliphatic solvent to carry the silicone polymer into the micro-pore structure of the wood. The overall composition is from 65 to 95% by weight aliphatic solvent, and the silicone based polymer is at least 5% by weight. The composition includes up to 5% by weight natural product oil in at least a biologically effective amount. The composition is prepared as follows: In a power stirred vessel is placed a volume of aliphatic solvent and the silicone based polymer is slowly added with stirring. When the desired volume of silicone based polymer is added, the desired volume of essential oil is slowly added to make up the final mixture. In this manner compositions of 5% Cedar oil in 65% Conosol 145/30% GT 5814; 75% Conosol 145/20% GT 5814; 80% Conosol 145/15% GT 5814 and 85% Conosol 145/and 10% GT 5814 are prepared. Samples of commercially available Oak, Maple, Yellow pine, and Western pine are treated by immersion in each mixture. Compositions containing a biologically effective amount of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil are prepared in a similar manner as that described above for cedar oil. A biologically effective amount means the minimum concentration necessary to obtain an observable decrease in biological damage between a treated sample and an untreated control sample of the same materials.

The invention is highly effective in treating wooded construction materials, and may also be beneficially used with materials containing wood fibers or any other cellulose of lignin containing construction material that requires protection against biological attack. The exact selection of treatment compositions and materials will vary depending on the specific protection needed and the availability of treatment materials near the source of the construction materials.

### EXAMPLE 4

Protection Against Termites

Filter paper comprising pine fibers was immersed in a composition prepared as set out in example 1 and comprising 85% Conosol 145, 10% GT 5814 and 5% Cedar oil and allowed to dry. An untreated control was also provided. The filter papers were placed in a test chamber and 50 worker Formosan Termites were placed in the chamber. After 24 hours the control paper has been almost totally consumed while the treated paper is untouched.

### EXAMPLE 5

Insecticidal Activity

Approximately 10 species of insects native to south Texas including German cockroach, cat fleas, mosquitoes, gypsy moths Japanese beetles, and horned beetles, were placed in a glass aquarium and exposed to a cube of wood approximately 2 cm on each side that was treated with a composition prepared as set out in example 1 and comprising 85% Conosol 145, 10% GT 5814 and 5% Cedar oil by immersion for one hour then air drying for 24 hours. After 24 hours, in each case the insects in the treated chamber were dead.

### EXAMPLE 6

Antifungal Activity

Side by side samples of flooring one piece treated by immersion in a composition prepared as set out in example 1 and comprising 85% Conosol 145, 10% GT 5814 and 5% Cedar oil with air drying and an untreated control from the same lot where placed in a damp area with active mildew growth. After 12 days the surface of the control untreated flooring was almost entirely covered with mildew and the treated flooring remained free of mildew.

### EXAMPLE 7

Insect Protection with Other Essential Oils

Cellulose fibers and wood samples are treated with a penetrating composition. Each sample is treated in a small treatment vessel by closing the wood to be treated in an enclosed treatment chamber, pulling a vacuum of 28 mm Hg on the treatment chamber for 30 minutes, releasing the vacuum and filling the treatment chamber with a treatment fluid having the composition as set out in the table below, venting the chamber to atmospheric pressure and holding for 1 hour. The treated sample is then cured for 12 hours. The treated samples set out below all show protection against biological damage against at least major pest over the untreated control of the same material. Treatment compositions were as follows:

TABLE 2

| Essential Oil | Percent oil | Percent GT5814 | Percent Conosol 145 |
|---|---|---|---|
| almond bitter oil | 5 | 10 | 85 |
| anise oil | 5 | 10 | 85 |
| basil oil | 5 | 10 | 85 |
| bay oil | 5 | 10 | 85 |
| caraway oil | 5 | 10 | 85 |
| cardamom oil | 5 | 10 | 85 |
| cedar oil | 5 | 10 | 85 |
| celery oil | 5 | 10 | 85 |
| chamomile oil | 5 | 10 | 85 |
| cinnamon oil | 5 | 10 | 85 |
| citronella oil | 5 | 10 | 85 |
| clove oil | 5 | 10 | 85 |
| coriander oil | 5 | 10 | 85 |
| cumin oil | 5 | 10 | 85 |
| dill oil | 5 | 10 | 85 |
| eucalyptus oil, | 5 | 10 | 85 |
| fennel oil | 5 | 10 | 85 |
| ginger oil | 5 | 10 | 85 |
| grapefruit oil, | 5 | 10 | 85 |
| lemon oil | 5 | 10 | 85 |
| lime oil | 5 | 10 | 85 |
| mint oil | 5 | 10 | 85 |
| parsley oil | 5 | 10 | 85 |
| peppermint oil | 5 | 10 | 85 |
| pepper oil | 5 | 10 | 85 |
| rose oil | 5 | 10 | 85 |
| spearmint oil | 5 | 10 | 85 |
| (menthol) | | | |
| sweet orange oil | 5 | 10 | 85 |
| thyme oil | 5 | 10 | 85 |
| turmeric oil | 5 | 10 | 85 |
| oil of wintergreen, | 5 | 10 | 85 |
| juniper oil | 5 | 10 | 85 |
| tall oil | 5 | 10 | 85 |
| pine oil | 5 | 10 | 85 |
| almond bitter oil | 1 | 19 | 80 |
| anise oil | 1 | 19 | 80 |
| basil oil | 1 | 19 | 80 |
| bay oil | 1 | 19 | 80 |
| caraway oil | 1 | 19 | 80 |
| cardamom oil | 1 | 19 | 80 |
| cedar oil | 1 | 19 | 80 |
| celery oil | 1 | 19 | 80 |
| chamomile oil | 1 | 19 | 80 |
| cinnamon oil | 1 | 19 | 80 |
| citronella oil | 1 | 19 | 80 |
| clove oil | 1 | 19 | 80 |
| coriander oil | 1 | 19 | 80 |
| cumin oil | 1 | 19 | 80 |
| dill oil | 1 | 19 | 80 |
| eucalyptus oil, | 1 | 19 | 80 |
| fennel oil | 1 | 19 | 80 |
| ginger oil | 1 | 19 | 80 |
| grapefruit oil, | 1 | 19 | 80 |
| lemon oil | 1 | 19 | 80 |
| lime oil | 1 | 19 | 80 |
| mint oil | 1 | 19 | 80 |
| parsley oil | 1 | 19 | 80 |
| peppermint oil | 1 | 19 | 80 |
| pepper oil | 1 | 19 | 80 |
| rose oil | 1 | 19 | 80 |
| spearmint oil | 1 | 19 | 80 |
| (menthol) | | | |
| sweet orange oil | 1 | 19 | 80 |
| thyme oil | 1 | 19 | 80 |
| turmeric oil | 1 | 19 | 80 |
| oil of wintergreen, | 1 | 19 | 80 |
| juniper oil | 1 | 19 | 80 |
| tall oil | 1 | 19 | 80 |
| pine oil | 1 | 19 | 80 |
| almond bitter oil | 3 | 12 | 85 |
| anise oil | 3 | 12 | 85 |
| basil oil | 3 | 12 | 85 |
| bay oil | 3 | 12 | 85 |
| caraway oil | 3 | 12 | 85 |
| cardamom oil | 3 | 12 | 85 |
| cedar oil | 3 | 12 | 85 |
| celery oil | 3 | 12 | 85 |
| chamomile oil | 3 | 12 | 85 |
| cinnamon oil | 3 | 12 | 85 |
| citronella oil | 3 | 12 | 85 |
| clove oil | 3 | 12 | 85 |
| coriander oil | 3 | 12 | 85 |
| cumin oil | 3 | 12 | 85 |
| dill oil | 3 | 12 | 85 |
| eucalyptus oil, | 3 | 12 | 85 |
| fennel oil | 3 | 12 | 85 |
| ginger oil | 3 | 12 | 85 |
| grapefruit oil, | 3 | 12 | 85 |
| lemon oil | 3 | 12 | 85 |
| lime oil | 3 | 12 | 85 |
| mint oil | 3 | 12 | 85 |
| parsley oil | 3 | 12 | 85 |
| peppermint oil | 3 | 12 | 85 |
| pepper oil | 3 | 12 | 85 |
| rose oil | 3 | 12 | 85 |
| spearmint oil | 3 | 12 | 85 |
| (menthol) | | | |
| sweet orange oil | 3 | 12 | 85 |
| thyme oil | 3 | 12 | 85 |
| turmeric oil | 3 | 12 | 85 |
| oil of wintergreen, | 3 | 12 | 85 |
| juniper oil | 3 | 12 | 85 |
| tall oil | 3 | 12 | 85 |
| pine oil | 3 | 12 | 85 |
| cedar oil | 1 | 4 | 95 |
| cedar oil | 5 | 20 | 75 |
| cedar oil | 5 | 25 | 70 |

### EXAMPLE 8

### Barrier Penetration

A test area was prepared by placing clean sand in a glass box, mixing the sand with distilled water (25% by weight), and spreading the mixture evenly over the floor of the box to give about 5 cm depth. Commercial 6 oz. (177.44 ml) plastic cups were seated in the sand so that the cups were seated in sand to simulate a structure and to make the termites tunnel under and through instead of over material to enter the structure. A choice test was set up by placing 2 inches (5.08 cm) of substrate in a cup and 2 inches of sand was placed in a control cup. Ten ml of water is added to the substrate or the sand and a ¾×¾″ seasoned wood block is placed on top of the substrate. Reticulitermes sp. subterranean termites were collected from an active colony on the Dorman Lake test site which is part of the Mississippi State University forest. A jar containing 1 gram of termites that have been in the jar for a minimum of seven days was inverted in the test area. The test area was kept dark, and behavior of the termites was observed. The termites were foraging and were creating tunnels throughout the testing arena. Replicates with a formulation according to the invention as described in Example 2 had no activity within the testing area up to twenty one days but the termites were foraging randomly in the sand under these substrates but not through them. There was also activity in several of the sand control areas.

### EXAMPLE 9

### Barrier Penetration Under No Choice Conditions

Termite foraging and movement was evaluated within a tunneling arena. The arena will be made from compartment storage containers that measure 6¼″×3½″ (15.875 cm×8.89 cm) and have five compartments that measure 1½″×3½″ (3.81 cm×8.89 cm) each. The containers were modified by drilling holes in the base of each end of each compartment to accept 2½″ (6.35 cm) long ¼″ OD Tygon tubing. Each compartment represented a tunneling arena and was connected to the base of two 55 ml polystyrene vials, each containing 20 g of silica sand and 4 ml of distilled water.

Reticulitermes sp. subterranean termites were collected from an active colony on the Dorman Lake test site which is part of the Mississippi State University forest. Two hundred (195 workers and 5 soldiers, to mimic natural caste proportions) termites were extracted from the wood, counted, and weighed. Three samples were taken to determine that 0.50 g of termites by weight would equal a proportion equal to the natural caste within a Retculitermes sp. colony. The termites were weighed and placed in one of the two vials.

Five replicates of each test and sand controls were constructed. All replicates were placed in a temperature controlled room that was kept unlighted for 4 weeks (28 days). A computer controlled temperature/humidity recorder was placed in the room to record the climatic data for the duration of the test. At the end of the period, the termite tunneling through the arena and feeding activity on wood was recorded. The results after 28 days resulted in no activity within the testing arena or in the wood block chamber the composition according to the invention and in the control sand chamber with no termite activity in the release chamber. However, replicates with vetiver tops, vetiver roots and mixed vitiver roots and tops had activity in release chamber, testing arena, and in the block chamber. Vetiver is a grass. Steam distillation of vetiver roots produces an essential oil reputed to have insect repellant properties.

We claim:

1. GT 5814GT 5814A method of treatment useful for preventing damage to construction materials by biological agents that comprises contacting the material with a composition comprising (1) from 65 to 95% of an aliphatic solvent composed primarily of C₇-C₁₆ straight chain aliphatic, cycloparaffinic and isoparaffinic hydrocarbons, that contains less than 0.5% aromatics; (2) a protective amount of at least one natural product oil selected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil; a synthetic natural product oil mimic that comprises at least one synthetically produced or isolated chemical identified as a component of a natural product oil elected from the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, oil of wintergreen, juniper oil, tall oil, pine oil and the balance further comprising at least one (3) silicone based polymer that forms a film in the presence of a catalyst and water comprising at least one component selected from the group consisting of (A) a copolymer of silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group; and (B) (MₐD_{b}T_{c}Q_{d})ₓ formula the following parameters apply: the ratio of a/(c+d) is between 0 and 4; the ratio of b to the rest is not subject to limitation provided the final crosslinker viscosity is below 350 cSt; and R is a generalized organic radical selected from: linear or branched hydrocarbon radicals of 1-8 carbons containing 0-1 degree of unsaturation, or phenyl, or trifluoropropyl radicals and at least one R group of each molecule must be a hydrolysable and (C) a catalyst; and maintaining the contact for a time sufficient to establish a change in the wood that provides a decrease in the hydrophilic quality of the pore structure of the wood relative to untreated wood from the same species.

2. The method of claim 21 comprising the aliphatic solvent (1) and the solvent selected is composed primarily of C₉-C₁₄ cycloparaffinic and isoparaffinic hydrocarbons.

3. The method of claim 21 comprising the aliphatic solvent (1) and the solvent selected is composed primarily of C₁₀-C₁₃ cycloparaffinic and isoparaffinic hydrocarbons.

4. The method of claim 21 comprising the aliphatic solvent (1) and the solvent selected is composed primarily of solvents capable of meeting applicable standards for a “food grade” classification.

5. The method of claim 21 comprising the aliphatic solvent (1) and the solvent selected is composed primarily of Conosol 145.

6. The method of claim 21 wherein an oil (2) is selected and further the oil is from the group consisting of cedar oil, cinnamon oil, citronella oil, clove oil, eucalyptus oil, juniper oil, tall oil, and pine oil.

7. The method of claim 21 wherein an oil (2) is selected and further the oil is cedar oil.

8. The method of claim 21 wherein R groups may be the same or different and each is a lower alkyl group of no more that four carbons.

9. The method of claim 21 that comprises a where all copolymer R groups are methyl.

10. The method of claim 21 that comprises a crosslinker having wherein an R group in an alkoxy group is an alkyl group each comprising from 1 to 4 carbon atoms.

11. The method of claim 30 that further comprises methyl groups at non-alkoxy position.

12. A method for preventing fungal or insect damage to cellulose fibers that comprises contacting the cellulose fibers with a composition that comprises a moisture reducing concentration of a silicone polymer comprising silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group for a time sufficient to alter the surface activity of the cellulose fibers and thereby reduce the absorption of water from the environment of the cellulose fibers so that the moisture of the cellulose fibers is less than twenty percent of the dry weight of the cellulose fibers when the cellulose fibers are exposed to 100% relative humidity.

13. The method of claim 32 wherein the silicone polymer is diluted with an aliphatic solvent composed primarily of C₇-C₁₆ straight chained paraffinic, cycloparaffinic and isoparaffinic hydrocarbons, that contains less than 0.5% aromatics.

14. The method of claim 32 wherein the silicone polymer is diluted with a white mineral oil.

15. The method of claim 32 wherein the silicone polymer is diluted with an aliphatic solvent composed primarily of C₉-C₁₄ straight chained paraffinic, cycloparaffinic and isoparaffinic hydrocarbons, that contains less than 0.5% aromatics.

16. The method of claim 32 wherein the silicone polymer is diluted with an aliphatic solvent composed primarily of C₁₀-C₁₃ straight chained paraffinic, cycloparaffinic and isoparaffinic hydrocarbons, that contains less than 0.5% aromatics.

17. The method of claim 32 wherein the silicone polymers are have R¹ groups that are methyl and R² groups that are not methyl, where 70 to 99% of the groups are R¹ and 1% to 10% of R² groups have a hydroxyl, alkoxy, or acyl group.

18. A composition of matter that comprises a silicone polymer comprising silicone units having the general formula: (MₐD_{b}T_{c}Q_{d})ₓ where M is R₃SiO_{1/2}—; D is R₂SiO—; T is RSiO_{3/2}—; Q is Si(O_{1/2})₄—; R is a generalized organic radical selected from: linear or branched hydrocarbon radicals of 1-8 carbons containing 0-1 degree of unsaturation, or phenyl, or trifluoropropyl radicals; a, b, c, d are real numbers and further provided the ratio of a/(c+d) is between 0 and 4; the ratio of b to the rest is not subject to limitation provided the final base viscosity is between 50-3500 cSt; and at least one R group of each molecule must be a hydrolysable group and from 65 to 99% of a diluent composed primarily of C₇-C₁₆ straight chained paraffinic, cycloparaffinic and isoparaffinic hydrocarbons, that contains less than 0.5% aromatics.

19. The composition of claim 38 wherein the silicone polymer is diluted with an aliphatic solvent composed primarily of C₉-C₁₄ straight chained paraffinic, cycloparaffinic and isoparaffinic hydrocarbons, that contains less than 0.5% aromatics.

20. The composition of claim 38 wherein the silicone polymer is diluted with an aliphatic solvent composed primarily of C₁₀-C₁₃ straight chained paraffinic, cycloparaffinic and isoparaffinic hydrocarbons, that contains less than 0.5% aromatics.
